(19) United States
(12) Patent Application Publication
(10) Pub. No.: US 2023/0137669 A1
(43) Pub. Date: May 4, 2023

(54) DETERMINING DRUG COMBINATIONS, SYNERGISTIC DRUG COMBINATION AND USE THEREOF IN PANCREATIC CANCER TREATMENT

(71) Applicant: Innoplexus AG, Eschborn (DE)

(72) Inventors: OM Sharma, Pune (IN); Ishita Mallick, Pune (IN); Amit Choudhari, Pune (IN); Vivekanand Patil, Satara (IN); Juergen Scheele, Buggingen (DE); Werner Seiz, Hofheim (DE); Dinesh Solanke, Nagpur (IN)

(73) Assignee: Innoplexus AG, Eschborn (DE)

(21) Appl. No.: 18/060,121

(22) Filed: Nov. 30, 2022

Related U.S. Application Data
(62) Division of application No. 17/377,899, filed on Jul. 16, 2021.
(60) Provisional application No. 63/052,996, filed on Jul. 17, 2020.

(51) Int. Cl. G16H 20/40; G16H 20/10

(52) U.S. Cl. CPC G16H 20/40; G16H 20/10

(57) ABSTRACT

A pharmaceutical composition comprising an effective amount of Dacomitinib as epidermal growth factor receptor (EGFR) inhibitor and a prostaglandin-Endoperoxide Synthase 2 (PTGS2) inhibitor, and one or more pharmaceutically acceptable excipients. A pharmaceutical composition comprising an effective amount of Dacomitinib as epidermal growth factor receptor (EGFR) inhibitor and an Adrenoceptor Beta 2 (ADRB2) inhibitor, and one or more pharmaceutically acceptable excipients. A method of treating pancreatic cancer, the method comprising the step of administering a therapeutically effective amount of the pharmaceutical composition to an individual in need thereof, the pharmaceutical composition comprising an effective amount of Dacomitinib as epidermal growth factor receptor (EGFR) inhibitor with a prostaglandin-Endoperoxide Synthase 2 (PTGS2) inhibitor or an Adrenoceptor Beta 2 (ADRB2) inhibitor, and one or more pharmaceutically acceptable excipients.

### CROSS-REFERENCE TO RELATED APPLICATIONS/INCORPORATION BY REFERENCE

This application claims the benefit of provisional patent application No. U.S. 63/052,996 as filed on Jul. 17, 2020 and is a divisional application of non-provisional patent application Ser. No. 17/377,899 as filed on Jul. 16 2021, the disclosures of which are incorporated herein by reference in their entireties.

### FIELD OF TECHNOLOGY

Certain embodiments of the disclosure relate to cancer treatment. More specifically, certain embodiments of the disclosure relate to determination of drug combinations and its use in pancreatic cancer treatment.

### BACKGROUND

Over the years, many advances, breakthroughs, and landmark discoveries have been witnessed in the diagnosis and treatment of cancer, a life-threatening disease involving abnormal cell growth with the potential to invade or spread to other parts of the human body. Pancreatic cancer remains the leading cause of death from solid malignancies worldwide.

Current treatment options, such as chemotherapy and targeted drugs, are not substantially effective in treating pancreatic cancer. Existing approved monotherapy drugs exhibit high chances of development of drug resistance as seeing the pathophysiology of the pancreatic cancer. There are many combination therapies in practice, but absence of an approved drug combination for pancreatic cancer treatment remains an underlying problem.

Further limitations and disadvantages of conventional and traditional approaches will become apparent to one of skill in the art, through comparison of such systems with some aspects of the present disclosure as set forth in the remainder of the present application with reference to the drawings.

### BRIEF SUMMARY OF THE DISCLOSURE

A method is disclosed for determination of drug combinations and its use in pancreatic cancer treatment, substantially as shown in and/or described in connection with at least one of the figures, as set forth more completely in the claims.

These and other advantages, aspects and novel features of the present disclosure, as well as details of an illustrated embodiment thereof, will be more fully understood from the following description and drawings.

### BRIEF DESCRIPTION OF SEVERAL VIEWS OF THE DRAWINGS

FIG. 1 is a block diagram that illustrates an exemplary system for determining drug combinations and use in pancreatic cancer treatment, in accordance with an exemplary embodiment of the disclosure.

FIG. 2 depicts MA plots for visual representation of genomic data before and after normalization, in accordance with an exemplary embodiment of the disclosure.

FIG. 3 depicts drug details, mechanism of action, and steps for synthesis reaction for Dacomitinib, in accordance with an exemplary embodiment of the disclosure.

FIGS. 4A and 4B depict flowcharts illustrating exemplary operations for determining drug combinations and use in pancreatic cancer treatment, in accordance with various exemplary embodiments of the disclosure.

FIG. 5 is a conceptual diagram illustrating an example of a hardware implementation for a system employing a processing system for determining drug combinations and use in pancreatic cancer treatment, in accordance with an exemplary embodiment of the disclosure.

### DETAILED DESCRIPTION OF THE DISCLOSURE

Certain embodiments of the disclosure relate to determination of drug combinations and use in pancreatic cancer treatment. Various embodiments of the disclosure provide a method for determining drug combinations, a method of treating pancreatic cancer, and a pharmaceutical composition, which correspond to a solution for an effective new therapy for advanced and metastatic pancreatic cancer. Currently, chances of development of drug resistance are very high for monotherapy drugs seeing the pathophysiology of Pancreatic cancer. The problem of drug resistance is one of the major problems for cancer treatment. The solution in the present disclosure has the potential to determine accurate, effective, and synergistic drug combination candidates from thousands of molecules to help researchers to fast track clinical trials, increase success rate of clinical trials, thereby alleviate the burden of pancreatic cancer (which has one of the highest mortality index) from patients, healthcare industry and other stakeholders, and increase the efficacy of the treatment and thus, overall survival of the patients suffering from it.

In accordance with various embodiments of the disclosure, a method may be provided for determining drug combinations and use in pancreatic cancer treatment. The method may include retrieving, by one or more processors, pancreatic cancer datasets from a plurality of data sources based on selected types of expression profiling. The method may further include determining, by the one or more processors, a set of feature genes based on differential gene expression analysis (i.e., based on transcriptomics analysis) of diseased samples and control samples in normalized pancreatic cancer datasets. The method may further include selecting, by the one or more processors, pancreatic cancer targets for combination analysis based on druggability and the determined set of feature genes. The method may further include determining, by the one or more processors, a plurality of synergistic target pairs based on node embedded clustering of the selected pancreatic cancer targets. One of each pair of target pair is an epidermal growth factor receptor inhibitor. The method may further include selecting, by the one or more processors, candidate pairs of drug combinations from a plurality of pairs of drug combinations based on a cumulative ranking score of each pair of drug combination and the plurality of synergistic target pairs. The method may further include determining, by the one or more processors, one or more sets of drug combinations based on prioritization of the candidate pairs of drug combinations, filtration of drug combinations of the epidermal growth factor receptor inhibitor, and external validation.

In accordance with an embodiment, the selected types of expression profiling correspond to at least expression profiling by high throughput sequencing and expression profiling by array.

In accordance with an embodiment, the method may further include normalizing, by the one or more processors, the retrieved pancreatic cancer datasets based on one or more statistical techniques. In accordance with an embodiment, the determined set of feature genes correspond to differentially expressed genes (DEG).

In accordance with an embodiment, the method may further include prioritizing, by the one or more processors, the determined set of feature genes based on one or more artificial intelligence (AI) and machine learning (ML) techniques. In accordance with an embodiment, the method may further include validating, by the one or more processors, the determined set of feature genes based on a transcriptomics analysis.

In accordance with an embodiment, the method may further include determining, by the one or more processors, a plurality of pancreatic cancer targets based on confirmation of clinical and approved drugs with respect to the determined set of feature genes.

In accordance with an embodiment, the selection of the pancreatic cancer targets from the determined plurality of pancreatic cancer targets is based on a relevancy score through preclinical data extracted from one or more databases.

In accordance with an embodiment, the plurality of synergistic target pairs is determined based on analysis of node embedded clustering of a protein-protein interactions (PPI) network.

In accordance with an embodiment, the method may further include determining, by the one or more processors, the plurality of pairs of drug combinations based on a plurality of permutation and combination generated for a first drug that corresponds to the epidermal growth factor receptor inhibitor and a plurality of second drugs that corresponds to each of the plurality of synergistic target pairs.

In accordance with an embodiment, the method may further include determining, by the one or more processors, a first plurality of scores for the candidate pairs of drug combinations and a second plurality of scores for the plurality of synergistic target pairs. In accordance with an embodiment, the cumulative ranking score is based on the first plurality of scores and the second plurality of scores.

In accordance with an embodiment, the first plurality of scores and the second plurality of scores correspond to one or more of a closeness centrality score, a betweenness centrality score, a pathway coverage score, a target coverage score, drug safety scores, a proximity score, a combination publication count score, a combination clinical trials count score, literature evidence-based scores, and target centrality scores in a PPI network.

In accordance with an embodiment, the prioritization of the candidate pairs of drug combinations is based on a multicriteria decision technique.

In accordance with another aspect of the disclosure, a method of treating pancreatic cancer is disclosed. The method comprises the step of administering a therapeutically effective amount of the pharmaceutical composition to an individual in need thereof. The pharmaceutical composition comprises an effective amount of Dacomitinib as epidermal growth factor receptor (EGFR) inhibitor and a prostaglandin-Endoperoxide Synthase 2 (PTGS2) inhibitor, and one or more pharmaceutically acceptable excipients.

In accordance with an embodiment, the PTGS2 inhibitor is selected from the group consisting of Sulindac, Meloxicam, Etodolac, Naproxen, Monobenzone, Etoricoxib, Rofecoxib, Celecoxib, or a pharmaceutically acceptable salt or prodrug thereof.

In accordance with an embodiment, the PTGS2 inhibitor inhibits upregulated PTGS2 expression, which in turn increases the therapeutic effect of Dacomitinib in treatment of pancreatic cancer.

In accordance with another aspect of the disclosure, a pharmaceutical composition, in part, is disclosed. The pharmaceutical composition comprises an effective amount of Dacomitinib as EGFR inhibitor and a PTGS2 inhibitor, and one or more pharmaceutically acceptable excipients.

In accordance with an embodiment, the PTGS2 inhibitor is selected from the group consisting of Sulindac, Meloxicam, Etodolac, Naproxen, Monobenzone, Etoricoxib, Rofecoxib, Celecoxib, or a pharmaceutically acceptable salt or prodrug thereof.

In accordance with an embodiment, the pharmaceutical composition is in the form of a combination product. In accordance with an embodiment, the PTGS2 inhibitor inhibits upregulated PTGS2 expression, which in turn increases the therapeutic effect of Dacomitinib in treatment of pancreatic cancer.

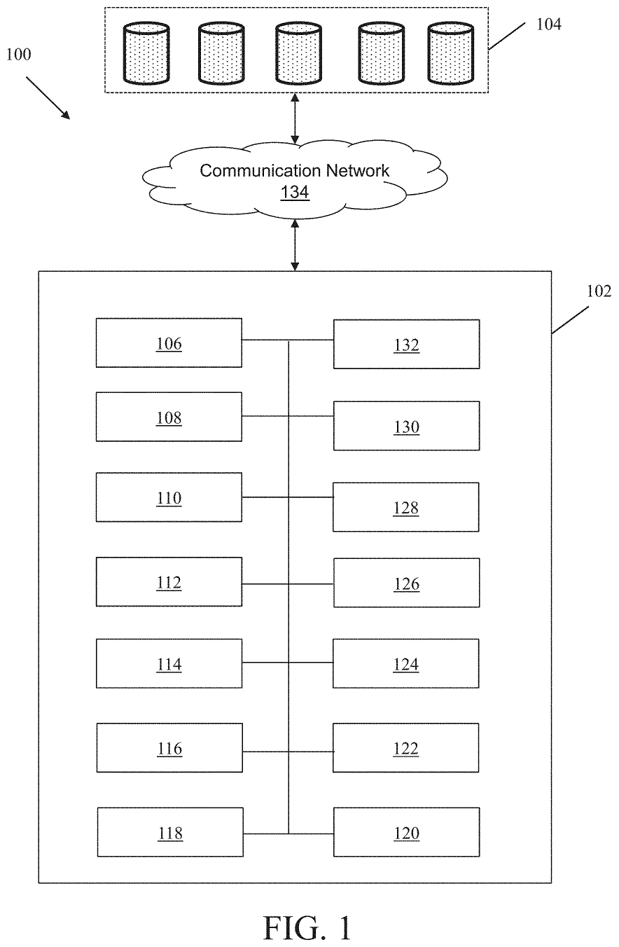

FIG. 1 is a block diagram that illustrates an exemplary system for determining drug combinations and use in pancreatic cancer treatment, in accordance with an exemplary embodiment of the disclosure. Referring to FIG. 1, a system 100 includes at least a computing device 102 and a plurality of data sources 104. The computing device χcomprises artificial intelligence (AI)/machine learning (ML) engine 106, and one or more processors, such as a processor 108, a dataset retrieval and normalization engine 110, a feature genes identification engine 112, a transcriptomics analysis engine 114, a target engine 116, a synergistic target engine 118, a drug combination engine 122, and a scoring engine 120. The computing device 102 further comprises a memory 124, a storage device 126, an input/output (I/O) device 128, a user interface 130, and a wireless transceiver 132. The plurality of data sources 104 are external or remote resources but communicatively coupled to the computing device 102 via a communication network 134.

In some embodiments of the disclosure, the AI/ML engine 106 may be integrated with other processors and engines to form an integrated system. In some embodiments of the disclosure, the one or more processors of the computing device 102 may be integrated with each other to form an integrated system. In some embodiments of the disclosure, as shown, the AI/ML engine 106 and the one or more processors may be distinct from each other. Other separation and/or combination of the various processing engines and entities of the exemplary system 100 illustrated in FIG. 1 may be done without departing from the spirit and scope of the various embodiments of the disclosure.

The plurality of data sources 104 may correspond to a plurality of public resources, such as servers, programs, and machines, that may store biological, biomedical, and pharmaceutical knowledge relevant to specific disease and may serve as a starting point for a trainable computational model, for example, an ML model. In accordance with an embodiment, the plurality of data sources 104 may provide pancreatic cancer datasets to the computing device 102 upon receiving an input from the computing device 102. The input may correspond to one or more types of expression profiling. Examples of such plurality of data sources 104 may include, but are not limited to, Gene Expression Omnibus (GEO) and Cancer Genome Atlas (TCGA) databases.

Notwithstanding, various types of the plurality of data sources 104, as exemplified above, should not be construed to be limiting, and various other types of plurality of data sources 104 may also be used, without deviation from the scope of the disclosure.

The AI/ML engine 106 may comprise suitable logic, circuitry, interfaces, and/or code that may be operable to implement AI and ML techniques in conjunction with the one or more processors. More specifically, the AI techniques, in conjunction with the one or more processors, may enable the computing device 102 to perform intellectual tasks, such as decision making, problem solving, perception and understanding human communication. The ML techniques, in conjunction with the one or more processors, may provide a set of tools that may improve discovery and decision making for well-specified questions with abundant, high-quality data. In accordance with an embodiment, the AI/ML engine 106 may implement the ML techniques in various processes of multi-omics data analysis, adverse event-based drug repurposing, network simulations to know non-obvious drugs exhibit in-direct connections with disease or target, safety profiling of drugs based on numbers and severity of adverse events, and drug combination prediction. All the standard parameters are confirmed while processing the datasets and generating an ML model by the AI/ML engine 106. For example, the AI/ML engine 106, in conjunction with the feature genes identification engine 112, may implement and execute AI/ML techniques, such as Random forest, Xgboost and decision tree, to analyze the datasets and provide desired results.

The processor 108 may comprise suitable logic, circuitry, interfaces, and/or code that may be operable to process and execute a set of instructions stored in the memory 124 or the storage device 126. In some embodiments, multiple processors and/or multiple buses may be used, as appropriate, along with multiple units and types of memory. Also, multiple processors, each providing portions of the necessary operations (for example, as a server cluster, a group of servers, or a multi-processor system), may be inter-connected and integrated. The processor 108 may be implemented based on several processor technologies known in the art. Examples of the processor may be an X86-based processor, a Reduced Instruction Set Computing (RISC) processor, an Application-Specific Integrated Circuit (ASIC) processor, a Complex Instruction Set Computing (CISC) processor, and/or other processors.

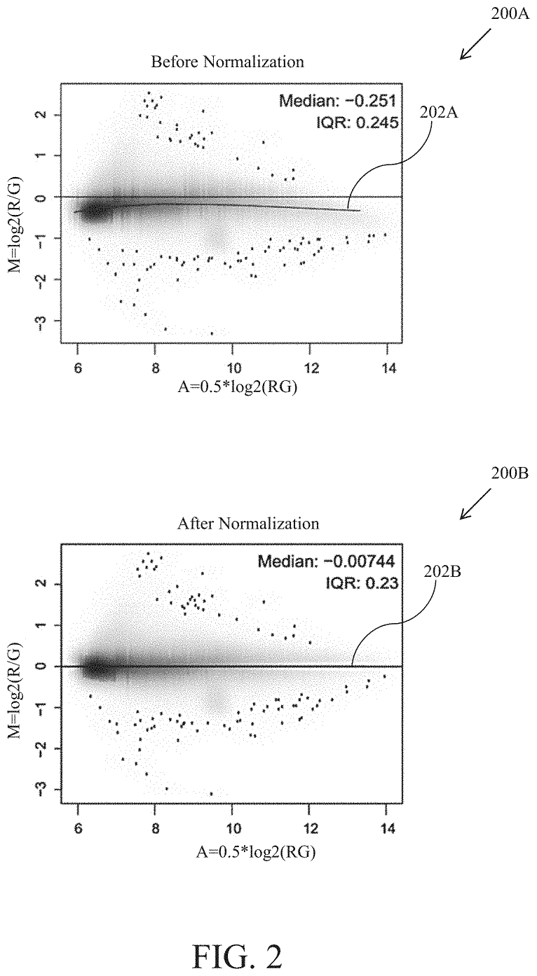

The dataset retrieval and normalization engine 110 may comprise suitable logic, circuitry, interfaces, and/or code that may be operable to retrieve pancreatic cancer datasets from the plurality of data sources 104 based on selected types of expression profiling. The dataset retrieval and normalization engine 110 may be further configured to normalize the retrieved pancreatic cancer datasets based on one or more statistical techniques. In accordance with an embodiment, expression data in the pancreatic cancer datasets may be crawled in an automated way through HTML based crawling. The retrieved pancreatic cancer datasets may be normalized using quantile normalization approach for normalizing gene expression counts across the sample and various tissue types, as illustrated in FIG. 2.

The feature genes identification engine 112 may comprise suitable logic, circuitry, interfaces, and/or code that may be operable to determine a set of feature genes based on differential gene expression analysis of the disease samples and control samples in the normalized pancreatic cancer datasets. The feature genes identification engine 112 may further prioritize the determined set of feature genes based on one or more AI and ML techniques.

In accordance with an exemplary embodiment, top significant differentially regulated genes (DEGs) may be determined based on the differentially expression values in comparison to the healthy volunteers. The differential gene expression analysis may be performed on two kinds of input data, such as raw data and normalized intensity data. All expression values of a gene may be normalized to logarithmic base 2, based on which logarithmic fold change (Log FC) may be calculated. The Log FC may correspond to a score which evaluates an average log-ratio between two groups. For example, based on fold change for a gene1 between disease samples and control samples, the differential expression, denoted by Log 2, may be calculated. As the first step, the expression values may be ensured in Log 2 form for both disease samples and control samples. Next, a mean may be calculated for each disease sample, denoted by “case mean” and likewise for control samples. Thereafter, a simple subtraction may be applied to the datasets wherein the control mean is subtracted from the case mean. Accordingly, a logarithmic base 2 fold change (Log2FC) may be achieved. Because all the values are in logarithmic form, subtraction is equivalent to division in normal mathematical values. In accordance with the exemplary embodiment, for each target family, the most significant DEGs or markers, as gene symbols, may be determined as follows:

| Target Family | Gene Symbols |
|---|---|
| GPCR | CXCR2, CCR5, CCR7, CHRM3, CXCR5, |
| | CALCRL, CCR2, PTGER4. |
| KINASE FAMILY | PIK3CG, PKM, CDK1, ERBB3, CCL2, BTK, |
| | TGFBR1. |
| TRANSCRIPTION | PDX1, VTN, WT1, SOX1, ID1. |
| FACTORS | |
| ENZYME | MMP9, PTGS2, CA9, MMP12, CEL, LOX, |
| | SPINK1, LDHA, SOD2, PLAT, DUOX2, MMP1. |
| ION CHANNEL | PKD2, CHRNA4, P2RX7, ANO1. |
| TRANSPORTER | SLC5A5 and NR1I2 |

The transcriptomics analysis engine 114 may comprise suitable logic, circuitry, interfaces, and/or code that may be operable to validate the determined set of feature genes based on transcriptomics analysis. In accordance with an embodiment, the transcriptomics analysis is the study of the transcriptome using high-throughput methods, such as microarray analysis. The transcriptome may correspond to the complete set of RNA transcripts that are produced by the genome, under specific circumstances or in a specific cell.

The target engine 116 may comprise suitable logic, circuitry, interfaces, and/or code that may be operable to determine the plurality of pancreatic cancer targets based on confirmation of clinical and approved drugs with respect to the determined set of feature genes. In accordance with an embodiment, the target engine 116 may generate higher ranks for targets associated with pancreatic cancer as well as present in the surface cellular compartments. Further, the target engine 116 may determine ranks for targets associated with pancreatic cancer based on druggability analysis. In accordance with an embodiment, the target engine 116 may select the pancreatic cancer targets from the determined plurality of pancreatic cancer targets based on a relevancy score through preclinical data extracted from one or more databases. In accordance with an embodiment, the target engine 116 may select pancreatic cancer targets for combination analysis based on druggability and the determined set of feature genes.

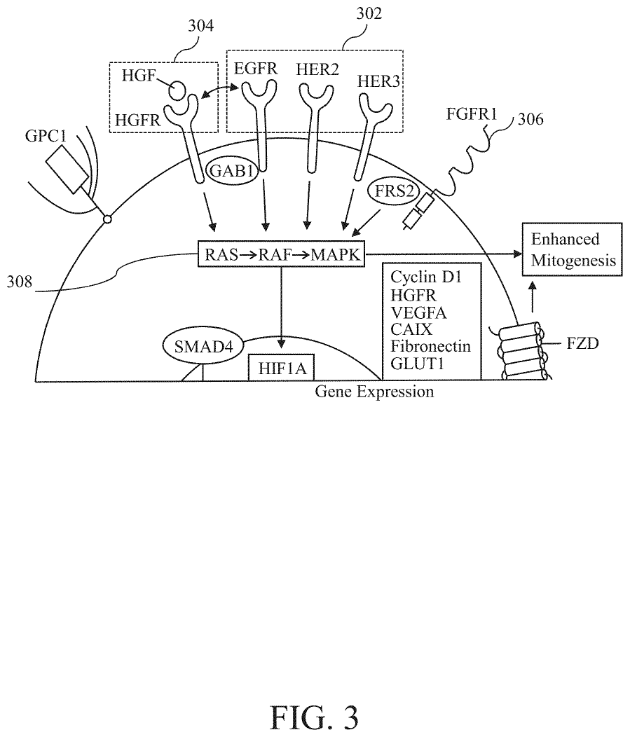

The synergistic target engine 118 may comprise suitable logic, circuitry, interfaces, and/or code that may be operable to determine a plurality of synergistic target pairs based on node embedded clustering of the selected pancreatic cancer targets. In accordance with an exemplary embodiment, one of each pair of target pairs is an epidermal growth factor receptor (EGFR) inhibitor, such as Dacomitinib. In accordance with an embodiment, the plurality of synergistic target pairs may be determined based on analysis of node embedded clustering of a protein-protein interactions (PPI) network. The mechanism of action and relevant details of Dacomitinib are described in detail in FIG. 3.

The scoring engine 120 may comprise suitable logic, circuitry, interfaces, and/or code that may be operable to determine a first plurality of scores for the candidate pairs of drug combinations and a second plurality of scores for the plurality of synergistic target pairs. In accordance with an exemplary embodiment, the scoring engine 120 may rank a drug combination based on corresponding mechanism of action. By way of various non-limiting examples, the first and second plurality of scores may correspond to one or more of a closeness centrality score, a betweenness centrality score, a pathway coverage score, a target coverage score, drug safety scores, a proximity score, a combination publication count score, a combination clinical trials count score, literature evidence-based scores, and target centrality scores in a PPI network.

The closeness centrality score may correspond to a score that is determined based on closeness of a target to other targets in the PPI network. The scoring engine 120 may calculate the closeness centrality score based on the sum of the path lengths from the given target to all other targets. In the context of target-target network, the closeness centrality score may indicate how close the given target is to other targets and hence plays an important role in the PPI network. The scoring engine 120 may calculate the closeness centrality score, that may be expressed as:

$$C ( x ) = \frac{N}{{\sum }_{y} d ( y , x )}$$

where N is total number of nodes, and
d(y, x) denotes the distance between node(y) and node(x).

The betweenness centrality score may correspond to a score that indicates how much a given node (hereinafter, denoted as “u”) is in-between other nodes. The betweenness centrality score may be measured based on the number of shortest paths (between any couple of nodes in the graphs) that passes through the target node “u”. The betweenness centrality score may be moderated by the total number of shortest paths existing between any couple of nodes of the graph. The target node “u” may have a high centrality if it appears in many shortest paths. The betweenness centrality score may be expressed as:

$$B ( u ) = {\sum }_{u \neq v \neq w} \frac{{σ}_{v , w} ( u )}{{σ}_{v , w}}$$

where σ_{v,w}(u) denotes the total number of shortest paths (between any couple of nodes in the graphs) that passes through the target node u, and
σ_{v,w} denotes total number of shortest paths existing between any couple of nodes of the graph.

The safety score may correspond to a drug safety score that may be calculated for a specific drug based on corresponding published adverse events. Thus, higher safety score indicates a better and safe drug. The safety score may be expressed as:

Σ(x [‘frequency’]*(Lethality factor*x[‘lethality’]+1))

where x is the reported adverse event for the drugs with Lethality factor=4 for moderate to severe events.

The pathway coverage score may correspond to a ratio of number of indication specific pathway covered by drugs in combination and number of all the pathway related to that indication. The pathway coverage score may be calculated using Jaccard index, based on the following expression:

$$J ( A , B ) = \frac{\vert A ⋂ B \vert}{\vert A ⋃ B \vert}$$

where A denotes pancreatic cancer pathway, and
B denotes U(Drug1 Pathway, Drug2 Pathway).

The proximity score may correspond to a distance-based score that may calculate distance between two targets in a target-target network. Thus, higher distance between two targets in combination indicates that the target combination is better, or the clustering is performed well. An efficient way to capture network proximity between a target (X) and a target (Y) is based in the z-score, expressed as

$$z = \frac{d - μ}{σ} ,$$

which relies on the shortest path lengths d(x, y) between target (X) and a target (Y), expressed as:

$$d ( X , Y ) = \frac{1}{‖ Y ‖} {\sum }_{y ∈ Y} {min}_{x ∈ X} d ( x , y )$$

The target coverage score may correspond to a ratio of number of indication specific targets covered by drugs in combination and number of all the targets related to that indication. The target coverage may be calculated using Jaccard index, expressed as:

$$J ( A , B ) = \frac{\vert A ⋂ B \vert}{\vert A ⋃ B \vert}$$

where A denotes pancreatic cancer targets, and
B denotes U(Drug1 Pathway, Drug2 Pathway).

The combination pub count score may correspond to a score that is based on a count of number of publications for the target pair in the combination, expressed as:

Publications (Drug1 target)∩Publications (Drug2 target)

The combination CT count score may correspond to a score that is based on a count of number of clinical trials for the target pair in the combination, expressed as:

Clinical trial (Drug1 target)∩Clinical trial (Drug2 target)

The confidence score may correspond to a score that is calculated based on the above scores to prioritize the drug combinations.

The drug combination engine 122 may comprise suitable logic, circuitry, interfaces, and/or code that may be operable to determine the plurality of pairs of drug combinations based on the plurality of permutation and combination generated for the first drug that corresponds to the epidermal growth factor receptor inhibitor and the plurality of second drugs that corresponds to each of the plurality of synergistic target pairs. The drug combination engine 122 may perform drug target mapping for the selected pancreatic cancer targets based on target expression pattern in the pancreatic cancer. The drug combination engine 122 may enlist top mapping drugs for further drug combination prediction. In accordance with an embodiment, the drug combination engine 122 may be configured to select candidate pairs of drug combinations from the plurality of pairs of drug combinations based on the cumulative ranking score of each pair of drug combination and the plurality of synergistic target pairs. In accordance with an embodiment, the drug combination engine 122 may be configured to prioritize the candidate pairs of drug combinations based on a multicriteria decision technique. One example of the multicriteria decision technique may be Analytic Hierarchy Process (AHP). In accordance with an embodiment, the drug combination engine 122 may be configured to determine the one or more sets of drug combinations based on prioritization of the candidate pairs of drug combinations, filtration of drug combinations of an epidermal growth factor receptor inhibitor, and external validation.

The memory 124 may comprise suitable logic, circuitry, and/or interfaces that may be operable to store a machine code and/or a computer program with at least one code section executable by the one or more processors, such as the processor 108. The memory 124 may be configured to store information within the computing device 102. In some embodiments, the memory 124 may be a volatile memory unit or units. In other embodiments, the memory 124 may be a non-volatile memory unit or units. In yet other embodiments, the memory 124 may be another form of computer-readable medium, such as a magnetic or optical disk. Examples of forms of implementation of the memory 124 may include, but are not limited to, a Random Access Memory (RAM), a Read Only Memory (ROM), a Hard Disk Drive (HDD), and/or a Secure Digital (SD) card.

The storage device 126 may be capable of providing mass storage to the computing device 102. In some embodiments, the storage device 126 may be or contain a computer-readable medium, such as a hard disk device, an optical disk device, or a tape device, a flash memory or other similar solid state memory device, or an array of devices, including devices in a storage area network or other configurations. A computer program product may be tangibly embodied in an information carrier. The information carrier may be a computer-readable or machine-readable medium, such as the memory 124 or the storage device 126. The computer program product may also contain instructions that, when executed, perform one or more methods, such as those described in the disclosure.

The I/O device 128 may comprise suitable logic, circuitry, interfaces, and/or code that may be configured to receive an input from a user and provide an output to the user of the computing device 102. The I/O device 128 may include various input and output devices that may be configured to facilitate a communication between the one or more processors in the computing device 102 and the user of the computing device 102. Examples of the input devices may include, but are not limited to, a hardware button on the computing device 102 to receive a selection or filtering criteria as the input from the user, a software button on the user interface 130 of the computing device 102, a camcorder, a touch screen, a microphone, and/or one or more sensors. Examples of the output devices may include, but are not limited to, a display on which the user interface 130 is presented, a projector screen, and/or a speaker.

The user interface 130 may comprise suitable logic, circuitry, and interfaces that may be configured to present the results, i.e. one or more sets of drug combinations, determined by the drug combination engine 122. The results may be presented in form of an audible, visual, tactile, or other output to the user, such as a researcher, a scientist, a principal investigator, and a health authority, associated with the computing device 102. As such, the user interface 130 may include, for example, a display, one or more switches, buttons, or keys (e.g., a keyboard or other function buttons), a mouse, and/or other input/output mechanisms. In an example embodiment, the user interface 130 may include a plurality of lights, a display, a speaker, a microphone, and/or the like. In some embodiments, the user interface 130 may also provide interface mechanisms that are generated on the display for facilitating user interaction. Thus, for example, the user interface 130 may be configured to provide interface consoles, web pages, web portals, drop down menus, buttons, and/or the like, and components thereof to facilitate user interaction.

The wireless transceiver 132 may comprise suitable logic, circuitry, interfaces, and/or code that may be operable to communicate with the other servers and electronic devices, via a communication network. The wireless transceiver 132 may implement known technologies to support wired or wireless communication of the computing device 102 with the communication network 134. The wireless transceiver 132 may include, but not limited to, an antenna, a radio frequency (RF) transceiver, one or more amplifiers, a tuner, one or more oscillators, a digital signal processor, a coder-decoder (CODEC) chipset, and/or a local buffer. The wireless transceiver 132 may communicate via wireless communication with networks, such as the Internet, an Intranet and/or a wireless network, such as a cellular telephone network. The wireless communication may use any of a plurality of communication standards, protocols and technologies, such as a Global System for Mobile Communications (GSM), Enhanced Data GSM Environment (EDGE), wideband code division multiple access (W-CDMA), code division multiple access (CDMA), time division multiple access (TDMA), Long Term Evolution (LTE), Bluetooth, Wireless Fidelity (Wi-Fi) (e.g., IEEE 802.11a, IEEE 802.11b, IEEE 802.11g and/or IEEE 802.11n), voice over Internet Protocol (VoIP), Wi-MAX, a protocol for email, instant messaging, and/or Short Message Service (SMS).

The communication network 134 may be any kind of network, or a combination of various networks, and it is shown illustrating exemplary communication that may occur between the plurality of data sources 104 and the computing device 102. For example, the communication network 134 may comprise one or more of a cable television network, the Internet, a satellite communication network, or a group of interconnected networks (for example, Wide Area Networks or WANs), such as the World Wide Web. Although one mode of communication network the communication network 134 is shown, the disclosure is not limited in this regard. Accordingly, other exemplary modes may comprise unidirectional or bidirectional distribution, such as packet-radio, and satellite networks.

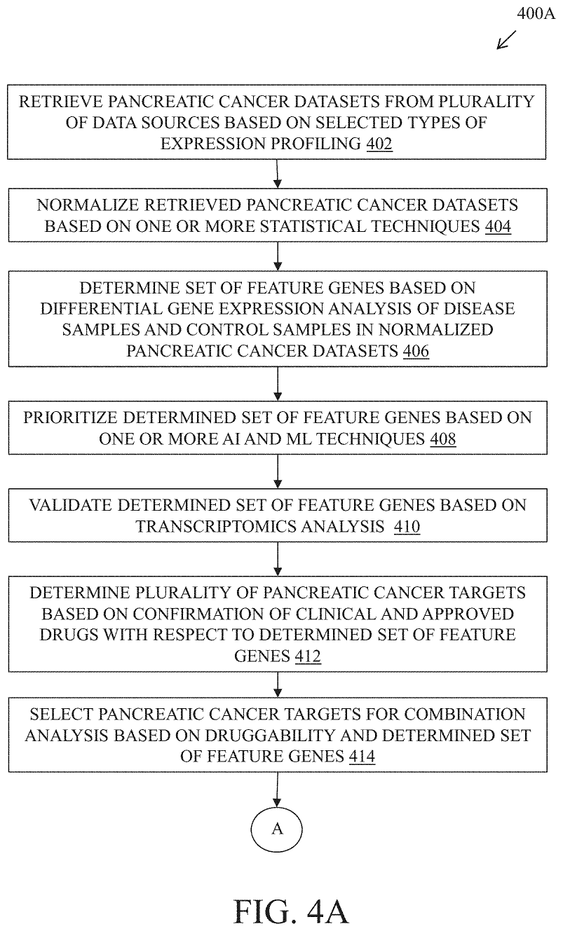

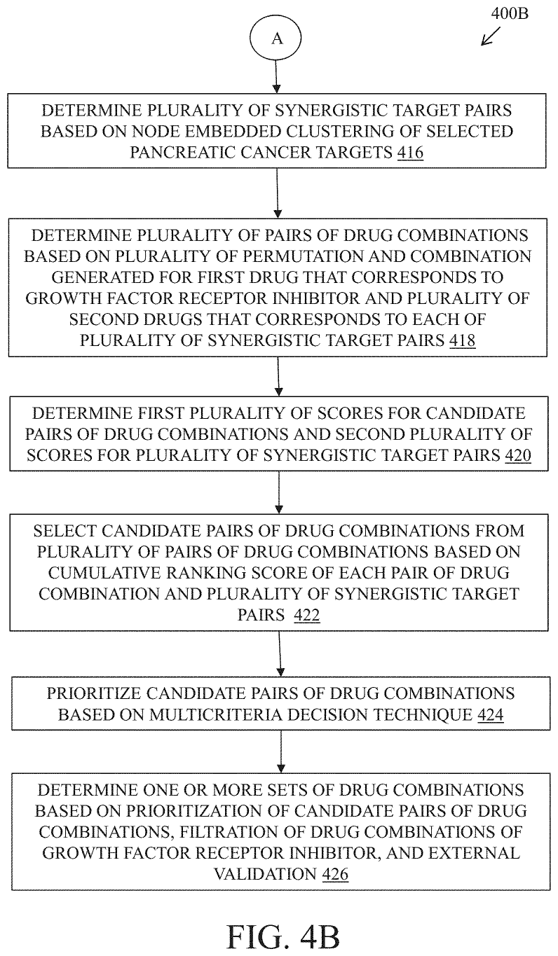

FIG. 2 depicts MA plots for visual representation of genomic data before and after normalization, in accordance with an exemplary embodiment of the disclosure. FIG. 2 is described in conjunction with FIG. 1 and FIGS. 4A and 4B.

Before normalization, various technical errors may occur during the microarray experimental procedure, such as, irregular spot printing, nonuniform intensity of the fluorescent compound, dusty arrays, purification errors, difference in efficiency of labelling via fluorescent dyes, hybridization efficiencies, and systematic biases in quantified expression levels. Such artifacts may have bearings on capturing data leading to different measurements of the same expression values. Hence, technical noises must be eliminated prior to a downstream analysis. Normalization reduces such potential systematic noises and ensures that there are no outliers or unnormalized datasets that may induce biases in the findings.

In accordance with an embodiment, the normalization may be performed based on quantile normalization. Quantile normalization may be a simple nonparametric normalization method initially proposed for single-channel arrays. Quantile normalization may be a between-array normalization method that makes the distribution of all arrays identical in statistical properties. The algorithm may map every expression value on each chip to the corresponding quantile of a reference distribution that is determined by pooling across distributions of all individual chips. The quantile normalization may be motivated by the idea that a quantile-quantile plot that shows the distribution of two data vectors is the same only if the plot is a straight diagonal line. The quantile normalization may explicitly assume that the distribution of gene expression measures is identical across the samples.

With reference to FIG. 2, two MA plots 200A and 200B are depicted to assess the performance of normalization methods by revealing systematic intensity-dependent effects in the measurements taken from two samples. An MA plot is an application of a Bland-Altman plot for visual representation of genomic data. The MA plot visualizes differences between measurements taken in two samples, by transforming the data onto M (log ratio) and A (mean average) scales, then plotting such values. In accordance with the embodiments of the present disclosure, the two samples, that is, query and reference samples, may be referred to as R and G (for the red and green colors used to represent Cy5 and Cy3 intensities in two-channel microarrays). The MA plots 200A and 200B may be prepared before and after normalization using the smoothScatter method which produces a smoothed color density representation of a scatter plot. Red line, as indicated by 202A and 202B in the MA plots 200A and 200B respectively in FIG. 2, is the lowess smoothed line to visually show the bias.

FIG. 3 depicts drug details, mechanism of action, and steps for synthesis reaction for Dacomitinib, in accordance with an exemplary embodiment of the disclosure.

Drug Details: Dacomitinib is a drug or medication, designed as (2E)-N-16-4-(piperidin -1-yl) but-2-enamide, and is an oral highly selective quinazalone part of the second-generation tyrosine kinase inhibitors which are characterized by the irreversible binding at the ATP domain of the epidermal growth factor receptor family kinase domains. Dacomitinib is indicated as the first-line treatment of patients with metastatic non-small cell lung cancer (NSCLC) with EGFR exon 19 deletion or exon 21 L858R substitution mutations as verified by an FDA-approved test. The structure and properties of Dacomitinib is illustrated and described below:

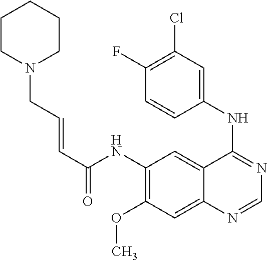 [SMILES: COc1cc2ncnc(Nc3ccc(F)c(Cl)c3)c2cc1NC(=O)/C=C/CN1CCCCC1]

The chemical formula is C24H25ClFN5O2 and the weight average is 469.939. The dosage form is tablet and strength may be one of 15 mg, 30 mg, or 45 mg. Cmax is at a dose of 45 mg orally once daily, the geometric mean [coefficient of variation (CV %)] Cmax was 108 ng/mL (35%). The area under the concentration-time curve (AUC) is 2213 ng·h/mL (35%). The time to reach maximum concentration occurred at approximately 6.0 h. The mean absolute bioavailability is 80% after oral administration. The volume of distribution is 1889 L (18%), and the drug is 98% bound to plasma proteins and is independent of drug concentrations from 250 ng/mL to 1000 ng/mL. The two most significant enzymes, i.e. CYP3A4 and CYP2D6, of cytochrome P450 enzymes are essential for the metabolism of Dacomitinib.

Mechanism of action: Dacomitinib is an irreversible small molecule inhibitor of the activity of the human epidermal growth factor receptor (EGFR) family (EGFR/HER1, HER2, and HER4) tyrosine kinases, denoted by dotted box 302. It achieves irreversible inhibition via covalent bonding to the cysteine residues in the catalytic domains of the HER receptors. Hepatocyte growth factor (HGF) and its receptor (HGFR), denoted by dotted box 304, ligand/receptor system controls essential cellular responses, such as cell proliferation and motility as well as morphogenesis and differentiation. Further, fibroblast growth factor receptors (FGFRs) 306 are a family of receptor tyrosine kinases expressed on the cell membrane that play crucial roles in both developmental and adult cells.

The affinity of dacomitinib has been shown to have an IC50 of 6 nmol/L. The ErbB or epidermal growth factor (EGF) family plays a role in tumor growth, metastasis, and treatment resistance by activating downstream signal transduction pathways, such as Ras-Raf-MAPK, denoted by 308, PLC gamma-PKC-NFkB, and PI3K/AKT through the tyrosine kinase-driven phosphorylation at the carboxy-terminus. Around 40% of cases show amplification of EGFR gene and 50% of the cases present the EGFRvIII mutation which represents a deletion that produces a continuous activation of the tyrosine kinase domain of the receptor.

ERBB2, EGFR are the targets with known mechanisms of action for Dacomitinib. Dacomitinib diminishes PDAC cell proliferation via inhibition of FOXM1 and its targets Aurora kinase B and cyclin B1. Dacomitinib induces apoptosis and potentiated radio-sensitivity via inhibition of the anti-apoptotic proteins surviving and MCL1. Dacomitinib shows attenuated cell migration and invasion through inhibition of the epithelial-to-mesenchymal transition (EMT) markers ZEB1, Snail and N-cadherin. EGFR is strongly associated with pancreatic cancer. High EGFR expression is significantly associated with distant metastasis (P=0.043) and severely decreases median overall survival time and recurrence-free survival time. High wild-type EGFR protein expression in tumor cells is a prognostic factor for reduced overall survival following pancreatic tumor resection, supporting a role for EGFR in identifying resected patients that may benefit from EGFR-targeted therapy.

Steps for synthesis reaction: First step may include performing cyclization on 2-amino-4-fluorobenzoic acid and formamide at high temperature, then successively performing nitration reaction, hydrogenation reduction, amidation reaction, methoxylation reaction and chlorination reaction. Final step includes splicing with 3-chloro-4-fluoroaniline to prepare the EGFR inhibitor Dacomitinib.

FIGS. 4A and 4B, depict flowcharts that collectively illustrate exemplary operations for determining drug combinations and use in pancreatic cancer treatment, in accordance with an embodiment of the disclosure. Flowcharts 400A and 400B of FIGS. 4A and 4B respectively, are described in conjunction with FIG. 1 to FIG. 3.

At step 402, pancreatic cancer datasets may be retrieved from the plurality of data sources 104 based on selected types of expression profiling. In accordance with an embodiment, the dataset retrieval and normalization engine 110 may be configured to retrieve pancreatic cancer datasets from the plurality of data sources 104 based on selected types of expression profiling. In accordance with an embodiment, the selected types of expression profiling may correspond to at least expression profiling by high throughput sequencing and expression profiling by array. For the retrieval, the plurality of data sources 104 may be accessed using the wireless transceiver 132, via the communication network 134.

In accordance with an embodiment, a filter may be applied by the user via the I/O device 128 to select the types of expression profiling. In such an embodiment, based on the applied filter, only human studies may be considered, and drug treated samples are removed. For example, the retrieved pancreatic cancer datasets may include four samples ID GSE15471, GSE16515, GSE28735 and TCGA-PAAD. In total, pancreatic cancer datasets comprise 294 disease samples (pretreated sample) and 104 control sample (healthy samples), which may be included for further differentially expression analysis.

At step 404, the retrieved pancreatic cancer datasets may be normalized based on one or more statistical techniques. In accordance with an embodiment, the dataset retrieval and normalization engine 110 may be configured to normalize the retrieved pancreatic cancer datasets based on the one or more statistical techniques. In accordance with an exemplary embodiment, the one or more statistical techniques may include quantile normalization approach for normalizing gene expression counts across the sample and various tissue types in the pancreatic cancer datasets. The quantile normalization approach for normalizing gene expression counts is described in detail in FIG. 2. In accordance with an embodiment, the expression data in the pancreatic cancer datasets may be crawled in an automated manner through HTML based crawling.

At step 406, a set of feature genes may be determined based on differential gene expression analysis of the disease samples and control samples in the normalized pancreatic cancer datasets. In accordance with an embodiment, the feature genes identification engine 112 may be configured to determine a set of feature genes based on the differential gene expression analysis of the disease samples and control samples in the normalized pancreatic cancer datasets. In accordance with an embodiment, the determined set of feature genes may correspond to differentially expressed genes (DEGs). In accordance with an exemplary embodiment, the feature genes identification engine 112 may determine 176 DEGs and validate them using publication count, as described in Table 1 below:

TABLE 1

| | | | PaCa |
|---|---|---|---|
| | Gene | | Publication |
| | Symbol | Target | Count |
| Target Name | (Gene Card) | family | (2016-2020) |
| Phosphatidylinositol 4,5-bisphosphate 3- | PIK3CG | Kinase | 223 |
| kinase catalytic subunit gamma isoform | | | |
| phosphatidylinositol-4,5-bisphosphate 3- | PIK3CD | Kinase | 212 |
| kinase catalytic subunit delta | | | |
| BCL2, apoptosis regulator | BCL2 | Non-IDG | 183 |
| Vimentin | VIM | Non-IDG | 172 |
| Matrix metalloproteinase-9 | MMP9 | Enzyme | 135 |
| Protein S100-A4 | S100A4 | Non-IDG | 125 |
| CEACAM7 protein, human | CEACAM7 | Non-IDG | 97 |
| Carcinoembryonic antigen-related cell | CEACAM5 | Non-IDG | 93 |
| adhesion molecule 5 | | | |
| mucin 1, cell surface associated | MUC1 | Non-IDG | 78 |
| tumor necrosis factor | TNF | Non-IDG | 71 |
| Interleukin-8 | CXCL8 | Non-IDG | 68 |
| Macrophage metalloelastase | MMP12 | Enzyme | 68 |
| Mesothelin | MSLN | Non-IDG | 64 |
| interleukin 18 | IL18 | Non-IDG | 55 |
| Mucin-16 | MUC16 | Non-IDG | 54 |
| C-X-C motif chemokine receptor 4 | CXCR4 | GPCR | 52 |
| prostaglandin-endoperoxide synthase 2 | PTGS2 | Enzyme | 51 |
| Fibronectin | FN1 | Non-IDG | 51 |
| Epithelial cell adhesion molecule | EPCAM | Non-IDG | 50 |
| Transthyretin | TTR | Non-IDG | 50 |
| Tissue factor | F3 | Non-IDG | 49 |
| hypoxia inducible factor 1 subunit alpha | HIF1A | Transcription Factor | 40 |
| sonic hedgehog | SHH | Non-IDG | 40 |
| Somatostatin | SST | Non-IDG | 38 |
| lactate dehydrogenase A | LDHA | Enzyme | 34 |
| catenin beta 1 | CTNNB1 | Non-IDG | 29 |
| Glucagon | GCG | Non-IDG | 27 |
| C-C motif chemokine 2 | CCL2 | Non-IDG | 26 |
| Stromelysin-1 | MMP3 | Non-IDG | 23 |
| erb-b2 receptor tyrosine kinase 3 | ERBB3 | Kinase | 22 |
| Cyclin-dependent kinase 1 | CDK1 | Kinase | 22 |
| C-X-C chemokine receptor type 2 | CXCR2 | GPCR | 21 |
| matrix metallopeptidase 7 | MMP7 | Enzyme | 21 |
| Neural cell adhesion molecule 1 | NCAM1 | Non-IDG | 21 |
| C-X-C motif chemokine ligand 10 | CXCL10 | Non-IDG | 19 |
| nuclear receptor subfamily 1 group I | NR1I2 | Nuclear Receptor | 18 |
| member 2 | | | |
| plasminogen activator, urokinase | PLAUR | Enzyme | 17 |
| receptor | | | |
| cathepsin B | CTSB | Enzyme | 17 |
| transforming growth factor beta | TGFBR1 | Kinase | 17 |
| receptor 1 | | | |
| heme oxygenase 1 | HMOX1 | Enzyme | 17 |
| killer cell lectin like receptor K1 | KLRK1 | Non-IDG | 17 |
| Keratin, type II cytoskeletal 7 | KRT7 | Non-IDG | 17 |
| Annexin A2 | ANXA2 | Non-IDG | 16 |
| Osteopontin | SPP1 | Non-IDG | 16 |
| carbonic anhydrase 9 | CA9 | Enzyme | 16 |
| mucin 2, oligomeric mucus/gel-forming | MUC2 | Non-IDG | 15 |
| DNA (cytosine-5)-methyltransferase 1 | DNMT1 | Enzyme | 14 |
| toll like receptor 2 | TLR2 | Non-IDG | 14 |
| CD163 molecule | CD163 | Non-IDG | 14 |
| Growth/differentiation factor 15 | GDF15 | Non-IDG | 13 |
| Galectin-1 | EGAES1 | Non-IDG | 13 |
| tissue factor pathway inhibitor | TFPI | Non-IDG | 13 |
| Plasminogen activator inhibitor 1 | SERPINE1 | Non-IDG | 13 |
| Platelet-derived growth factor receptor | PDGFRB | Kinase | 12 |
| beta | | | |
| Amphiregulin | AREG | Non-IDG | 12 |
| DNA topoisomerase II beta | TOP2B | Enzyme | 12 |
| Integrin alpha-M | ITGAM | Non-IDG | 12 |
| cholecystokinin B receptor | CCKBR | GPCR | 11 |
| Fas ligand | FASLG | Non-IDG | 11 |
| ephrin A2 | EFNA2 | Non-IDG | 11 |
| EPH receptor A2 | EPHA2 | Kinase | 11 |
| insulin like growth factor binding | IGFBP3 | Non-IDG | 11 |
| protein 3 | | | |
| integrin subunit beta 2 | ITGB2 | Non-IDG | 11 |
| glycoprotein nmb | GPNMB | Non-IDG | 11 |
| matrix metallopeptidase 1 | MMP1 | Enzyme | 10 |
| dickkopf WNT signaling pathway | DKK1 | Non-IDG | 10 |
| inhibitor 1 | | | |
| Macrophage mannose receptor 1 | MRC1 | Non-IDG | 10 |
| toll like receptor 7 | TLR7 | Non-IDG | 9 |
| C-C motif chemokine receptor 2 | CCR2 | GPCR | 9 |
| protein tyrosine phosphatase, receptor | PTPRC | Enzyme | 9 |
| type C | | | |
| Superoxide dismutase [Mn], | SOD2 | Enzyme | 8 |
| mitochondrial | | | |
| LIF, interleukin 6 family cytokine | LIF | Non-IDG | 8 |
| UDP-glucuronosyltransferase 1-1 | UGT1A1 | Enzyme | 8 |
| aryl hydrocarbon receptor | AHR | Transcription Factor | 8 |
| claudin 18 | CLDN18 | Non-IDG | 7 |
| Syndecan-1 | SDC1 | Non-IDG | 7 |
| T-lymphocyte activation antigen CD80 | CD80 | Non-IDG | 7 |
| Bile salt-activated lipase | CEL | Enzyme | 7 |
| macrophage stimulating 1 receptor | MST1R | Kinase | 7 |
| Neurotensin receptor type 1 | NTSR1 | GPCR | 7 |
| collagen type I alpha 1 chain | COL1A1 | Non-IDG | 7 |
| Vascular endothelial growth factor C | VEGFC | Non-IDG | 6 |
| Proteinase-activated receptor 1 | F2R | GPCR | 6 |
| Intermediate conductance calcium- | KCNN4 | Ion Channel | 6 |
| activated potassium channel protein 4 | | | |
| Bruton tyrosine kinase | BTK | Kinase | 6 |
| C-X-C chemokine receptor type 1 | CXCR1 | GPCR | 6 |
| Trefoil factor 1 | TFF1 | Non-IDG | 6 |
| CD86 molecule | CD86 | Non-IDG | 6 |
| carboxypeptidase A1 | CPA1 | Enzyme | 6 |
| FCGR3B protein, human | FCGR3A | Non-IDG | 6 |
| Lymphatic vessel endothelial hyaluronic | LYVE1 | Non-IDG | 6 |
| acid receptor 1 | | | |
| Lumican | LUM | Non-IDG | 5 |
| Urokinase-type plasminogen activator | PLAU | Enzyme | 5 |
| G protein-coupled receptor class C group | GPRC5A | GPCR | 5 |
| 5 member A | | | |
| P2X purinoceptor 7 | P2RX7 | Ion Channel | 5 |
| chymotrypsin C | CTRC | Enzyme | 5 |
| cytochrome P450 family 3 subfamily A | CYP3A5 | Enzyme | 5 |
| member 5 | | | |
| serpin family A member 5 | SERPINA5 | Enzyme | 5 |
| gap junction protein alpha 1 | GJA1 | Non-IDG | 4 |
| integrin subunit alpha V | ITGAV | Non-IDG | 4 |
| solute carrier family 15 member 1 | SLC15A1 | Transporter | 4 |
| mitogen-activated protein kinase kinase | MAP4K1 | Kinase | 4 |
| kinase kinase 1 | | | |
| C-C chemokine receptor type 5 | CCR5 | GPCR | 4 |
| ADAM metallopeptidase domain 9 | ADAM9 | Enzyme | 4 |
| anoctamin 1 | ANO1 | Ion Channel | 4 |
| carboxypeptidase B1 | CPB1 | Enzyme | 4 |
| guanylate cyclase 2C | GUCY2C | Kinase | 4 |
| Filamin-A | FLNA | Non-IDG | 4 |
| somatostatin receptor 5 | SSTR5 | GPCR | 4 |
| Actin, cytoplasmic 1 | ACTB | Non-IDG | 4 |
| C-type lectin domain containing 7A | CLEC7A | Non-IDG | 4 |
| matrix metallopeptidase 13 | MMP13 | Enzyme | 4 |
| Monocyte differentiation antigen CD14 | CD14 | Non-IDG | 4 |
| Wnt family member 1 | WNT1 | Non-IDG | 4 |
| Fas cell surface death receptor | FAS | Non-IDG | 3 |
| Amylin | IAPP | Non-IDG | 3 |
| S100 calcium binding protein A6 | S100A6 | Non-IDG | 3 |
| Vitronectin | VTN | Transcription Factor | 3 |
| Trefoil factor 2 | TFF2 | Non-IDG | 3 |
| Caspase-1 | CASP1 | Enzyme | 3 |
| integrin subunit alpha 5 | ITGA5 | Non-IDG | 3 |
| invariant chain | CD74 | Non-IDG | 3 |
| Regucalcin | RGN | Non-IDG | 3 |
| matrix metallopeptidase 10 | MMP10 | Non-IDG | 3 |
| mitogen-activated protein kinase kinase | MAP4K4 | Kinase | 3 |
| kinase kinase 4 | | | |
| progestagen associated endometrial | PAEP | Non-IDG | 3 |
| protein | | | |
| plasminogen activator, tissue type | PLAT | Enzyme | 2 |
| Mothers against decapentaplegic | SMAD7 | Transcription Factor | 2 |
| homolog 7 | | | |
| NADPH oxidase 4 | NOX4 | Enzyme | 2 |
| dipeptidase 1 | DPEP1 | Enzyme | 2 |
| serpin family F member 1 | SERPINF1 | Non-IDG | 2 |
| DCN protein, human | DCN | Non-IDG | 2 |
| Keratin, type II cytoskeletal 8 | KRT8 | Non-IDG | 2 |
| somatostatin receptor 1 | SSTR1 | GPCR | 2 |
| Versican | VCAN | Non-IDG | 2 |
| protein kinase C beta | PRKCB | Kinase | 2 |
| Receptor-type tyrosine-protein kinase | FLT3 | Kinase | 2 |
| FLT3 | | | |
| NUAK family kinase 1 | NUAK1 | Kinase | 2 |
| serine protease 3 | PRSS3 | Enzyme | 2 |
| secreted frizzled related protein 4 | SFRP4 | Non-IDG | 2 |
| HLA class I histocompatibility antigen, | HLA-B | Non-IDG | 2 |
| B-7 alpha chain | | | |
| NIMA related kinase 2 | NEK2 | Kinase | 2 |
| cholinergic receptor nicotinic alpha 4 | CHRNA4 | Ion Channel | 2 |
| subunit | | | |
| Cannabinoid receptor 2 | CNR2 | GPCR | 2 |
| C-C motif chemokine ligand 18 | CCL18 | Non-IDG | 2 |
| cytochrome P450 family 1 subfamily B | CYP1B1 | Non-IDG | 2 |
| member 1 | | | |
| Cytochrome b-245 heavy chain | CYBB | Ion Channel | 2 |
| P2X purinoceptor 5 | P2RX5 | Ion Channel | 2 |
| glycine N-methyltransferase | GNMT | Enzyme | 2 |
| selectin L | SELL | Non-IDG | 2 |
| Procollagen-lysine,2-oxoglutarate 5- | PLOD2 | Enzyme | 2 |
| dioxygenase 2 | | | |
| alpha-2-glycoprotein 1, zinc-binding | AZGP1 | Non-IDG | 2 |
| solute carrier organic anion transporter | SLCO1B3 | Transporter | 1 |
| family member 1B3 | | | |
| S100 calcium binding protein A2 | S100A2 | Non-IDG | 1 |
| Activin receptor type-1 | ACVR1 | Kinase | 1 |
| Pyruvate kinase PKM | PKM | Kinase | 1 |
| Complement C3 | C3 | Non-IDG | 1 |
| Alpha-1-antichymotrypsin | SERPINA3 | Non-IDG | 1 |
| Aldo-keto reductase family 1 member | AKR1B10 | Enzyme | 1 |
| B10 | | | |
| Tumor protein p73 | TP73 | Transcription Factor | 1 |
| mitogen-activated protein kinase kinase | MAP4K5 | Kinase | 1 |
| kinase kinase 5 | | | |
| cytochrome P450 family 24 subfamily | CYP24A1 | Enzyme | 1 |
| A member 1 | | | |
| BR serine/threonine kinase 2 | BRSK2 | Kinase | 1 |
| CCAAT/enhancer-binding protein beta | CEBPB | Transcription Factor | 1 |
| Insulin-like growth factor-binding | IGFBP7 | Non-IDG | 1 |
| protein 7 | | | |
| calcitonin receptor like receptor | CALCRL | GPCR | 1 |
| collagen type VI alpha 3 chain | COL6A3 | Non-IDG | 1 |
| Tumor necrosis factor receptor | TNFRSF11B | Non-IDG | 1 |
| superfamily member 11B | | | |
| S-adenosylmethionine synthase isoform | MAT1A | Enzyme | 1 |
| type-1 | | | |
| Lithostathine-1-alpha | REG1A | Non-IDG | 1 |
| toll like receptor 8 | TLR8 | Non-IDG | 1 |
| Transgelin | TAGLN | Non-IDG | 1 |
| Cellular retinoic acid-binding protein 2 | CRABP2 | Non-IDG | 1 |
| Arachidonate 5-lipoxygenase-activating | ALOX5AP | Enzyme | 1 |
| protein | | | |
| lysyl oxidase | LOX | Enzyme | 1 |
| NADPH oxidase 1 | NOX1 | Enzyme | 1 |

At step 408, the determined set of feature genes may be prioritized based on one or more AI and ML techniques. In accordance with an embodiment, the feature genes identification engine 112 in conjunction with the AI/ML engine 106, may be configured to prioritize the determined set of feature genes based on one or more AI and ML techniques. Non-limiting examples of such AI/ML techniques may include, Random Forest, Xgboost and Decision tree, known in the art.

In accordance with an exemplary embodiment, the Random Forest algorithm may be applied to identify the most important feature genes for pancreatic cancer. According to the Random Forest algorithm, a Random Forest classifier may use a splitting function, hereinafter referred to as “Gini index”, to determine which attribute to split on during tree learning phase. The Gini index may measure the level of impurity/inequality of the samples assigned to a node based on a split at its parent node. For example, under binary classification case, the Gini index may be defined as:

Gk=2p(1−p)

where p represents the fraction of positive examples assigned to a certain node k, and (1−p) represents the fraction of negative examples.

The purity of a node is indicated by a smaller Gini index. Every time a split of a node is made using a certain feature attribute, the Gini value for the two descendant nodes is less than the parent node. A feature's Gini importance value in a single tree may be then defined as the sum of the Gini index reduction (from parent to children) over all nodes in which the specific feature is used to split. The overall importance in the forest may be defined as the sum or the average of its importance value among all trees in the forest. Based on the same principle, the feature genes identification engine 112 in conjunction with the AI/ML engine 106, may be configured to prioritize the determined set of feature genes.

At step 410, the determined set of feature genes may be validated based on transcriptomics analysis. In accordance with an embodiment, the transcriptomics analysis engine 114 may be configured to validate the determined set of feature genes based on the transcriptomics analysis. In accordance with an exemplary embodiment, the transcriptomics analysis analyzes the complete set of RNA transcripts that may be produced by the genome, under specific circumstances or in a specific cell, using high-throughput methods, such as microarray analysis. Consequently, by analyzing the entire collection of RNA sequences in a cell (the transcriptome), it may be determined when and where each gene is turned on or off in the cells and tissues of a subject, such as a patient.

At step 412, a plurality of pancreatic cancer targets may be determined based on confirmation of clinical and approved drugs with respect to the determined set of feature genes. In accordance with an embodiment, the target engine 116 may be configured to determine the plurality of pancreatic cancer targets based on confirmation of clinical and approved drugs with respect to the determined set of feature genes. The pancreatic cancer targets correspond to protein coding genes, which when overexpressed or upregulated cause cancer cells to divide more rapidly. Various non-limiting examples of pancreatic cancer targets may include EGFR, Prostaglandin-Endoperoxide Synthase 2 (PTGS2), Adrenoceptor Beta 2 (ADRB2), and Vascular endothelial growth factor A (VEGF-A). In accordance with an embodiment, the target engine 116 may generate higher ranks for targets associated with pancreatic cancer as well as present in the surface cellular compartments. Further, the target engine 116 may determine ranks for targets associated with pancreatic cancer based on druggability analysis.

In accordance with an exemplary embodiment, the target engine 116 may cross-check available clinical/approved drugs against 176 DEGs (considered as druggable proteins). Based on the cross-checking of the available clinical/approved drugs against 176 DEGs, the target engine 116 may result in shortlisting of 60 protein targets. Various algorithms, such as Pagerank, community ranking, and Hyper-induced Topic Search (HITS), may be used to prioritize important targets for pancreatic cancer.

At step 414, pancreatic cancer targets may be selected for combination analysis based on druggability and the determined set of feature genes. In accordance with an embodiment, the target engine 116 may be configured to select pancreatic cancer targets for combination analysis based on druggability and the determined set of feature genes. In accordance with an embodiment, the target engine 116 may select the pancreatic cancer targets from the determined plurality of pancreatic cancer targets based on a relevancy score through preclinical data extracted from one or more databases.

For example, with reference to the validated set of 176 DEGs from transcriptomics analysis, direct 54 interactors with druggability may be determined. Based on relevancy through preclinical studies (literature data), top 54 may be selected as an input target for the combination analysis. In accordance with an embodiment, the selected pancreatic cancer targets may be scanned for gene ontology, such as biological process, cellular component, molecular function, to perform disease enrichment and pathway enrichment.

At step 416, a plurality of synergistic target pairs may be determined based on node embedded clustering of the selected pancreatic cancer targets. In accordance with an embodiment, the synergistic target engine 118 may be configured to determine a plurality of synergistic target pairs based on node embedded clustering of the selected pancreatic cancer targets. In accordance with an exemplary embodiment, one of each pair of target pair is an epidermal growth factor receptor, i.e. EGFR. In accordance with an embodiment, the plurality of synergistic target pairs may be determined based on analysis of node embedded clustering of a protein-protein interactions (PPI) network.

At step 418, a plurality of pairs of drug combinations may be determined based on a plurality of permutation and combination generated for a first drug that corresponds to the epidermal growth factor receptor inhibitor and a plurality of second drugs that corresponds to each of the plurality of synergistic target pairs. In accordance with an embodiment, the drug combination engine 122 may be configured to determine the plurality of pairs of drug combinations based on the plurality of permutation and combination generated for the first drug that corresponds to the epidermal growth factor receptor inhibitor and the plurality of second drugs that corresponds to each of the plurality of synergistic target pairs.

In accordance with an embodiment, the drug combination engine 122 may perform drug target mapping for the selected pancreatic cancer targets based on target expression pattern in the pancreatic cancer. The drug combination engine 122 may enlist top mapping drugs for further drug combination prediction. Further, multiple permutation and combination may be generated for each drug and corresponding targets in pairs. For example, a list of 40K pairs of drug combinations may be generated based on the plurality of permutation and combination for the first drug that corresponds to the epidermal growth factor receptor inhibitor and the plurality of second drugs that corresponds to each of the plurality of synergistic target pairs.

At step 420, a first plurality of scores for the candidate pairs of drug combinations and a second plurality of scores for the plurality of synergistic target pairs may be determined. In accordance with an embodiment, the scoring engine 120 may be configured to determine the first plurality of scores for the candidate pairs of drug combinations and the second plurality of scores for the plurality of synergistic target pairs. The first and the second plurality of scores may correspond to one or more of a closeness centrality score, a betweenness centrality score, a pathway coverage score, a target coverage score, drug safety scores, a proximity score, a combination publication count score, a combination clinical trials count score, literature evidence-based scores, and target centrality scores in the PPI network. The first and the second plurality of scores have been described in detail in FIG. 1.

In accordance with an exemplary embodiment, the scoring engine 120 may rank a drug combination based on corresponding mechanism of action. For example, network analysis-based ranking may be performed for each combination. High coverage of the pathway may result in a higher rank and high number of pathway intersections may be considered for penalty. In another example, drug synergy score may be calculated based on adverse events (AE) and toxicity. Survival probability may be also calculated based on target combination.

At step 422, candidate pairs of drug combinations may be selected from the plurality of pairs of drug combinations based on a cumulative ranking score of each pair of drug combination and the plurality of synergistic target pairs. In accordance with an embodiment, the drug combination engine 122 may be configured to select candidate pairs of drug combinations from the plurality of pairs of drug combinations based on the cumulative ranking score of each pair of drug combination and the plurality of synergistic target pairs.

In accordance with an exemplary embodiment, the cumulative ranking score may be based on the first plurality of scores and the second plurality of scores. In accordance with an embodiment, evidence score associated with, for example clinical trials (CT), publications, and grants, may be calculated and merged with the cumulative score. Accordingly, top ranked combinations may be proposed for possible drug combination for pancreatic cancer. For example, a candidate pair of drug combination is selected based on the safety score of 96.92 for the pair of drug combination, i.e. Dacomitinib and Monobenzone, and the synergistic target pair, i.e. EGFR and PTGS2, as shown in Table 2 below, which includes further such example drug combinations:

TABLE 2

| | | | | PaCa Combination | | |
|---|---|---|---|---|---|---|
| Target 1 | Target 2 | Drug 1 | Drug 2 | Patent | Clinical trial | Safety score |
| EGFR | PTGS2 | Dacomitinib | Monobenzone | No | No | 96.92 |
| EGFR | PTGS2 | Dacomitinib | Naproxen | No | No | 78.87 |
| EGFR | PTGS2 | Dacomitinib | Etoricoxib | No | No | 78.13 |
| EGFR | PTGS2 | Dacomitinib | Etodolac | No | No | 75.69 |
| EGFR | PTGS2 | Dacomitinib | Meloxicam | No | No | 74.86 |
| EGFR | PTGS2 | Dacomitinib | Sulindac | No | No | 74.41 |
| EGFR | PTGS2 | Dacomitinib | Celecoxib | No | No | 69.51 |
| EGFR | PTGS2 | Dacomitinib | Rofecoxib | No | No | 62.95 |
| EGFR | ADRB2 | Dacomitinib | Doxofylline | No | No | 86.9 |
| EGFR | ADRB2 | Dacomitinib | Nebivolol | No | No | 77.16 |
| EGFR | ADRB2 | Dacomitinib | Timolol | No | No | 76.88 |
| EGFR | ADRB2 | Dacomitinib | Salmeterol | No | No | 73.44 |
| EGFR | ADRB2 | Dacomitinib | Nadolol | No | No | 71.91 |
| EGFR | ADRB2 | Dacomitinib | Octreotide | No | No | 71.12 |
| EGFR | ADRB2 | Dacomitinib | Atenolol | No | No | 69.47 |
| EGFR | ADRB2 | Dacomitinib | Carvedilol | No | No | 69.36 |
| EGFR | ADRB2 | Dacomitinib | Metoprolol | No | No | 67.63 |
| EGFR | ADRB2 | Dacomitinib | Propafenone | No | No | 64.67 |
| EGFR | ADRB2 | Dacomitinib | Propranolol | No | No | 63.31 |
| EGFR | VEGF-A | Dacomitinib | Bevacizumab | No | Yes (Target | 69.92 |
| | | | | | combination) | |
| EGFR | VEGF-A | Dacomitinib | Enoxaparin | No | Yes (Target | 65.9 |
| | | | | | combination) | |

At step 122, the candidate pairs of drug combinations may be prioritized based on a multicriteria decision technique. In accordance with an embodiment, the drug combination engine 122 may be configured to prioritize the candidate pairs of drug combinations based on a multicriteria decision technique. One example of the multicriteria decision technique may be Analytic Hierarchy Process (AHP), known in the art.

At step 426, one or more sets of drug combinations may be determined based on prioritization of the candidate pairs of drug combinations, filtration of drug combinations of an epidermal growth factor receptor inhibitor, and external validation. In accordance with an embodiment, the drug combination engine 122 may be configured to determine the one or more sets of drug combinations based on prioritization of the candidate pairs of drug combinations, filtration of drug combinations of the epidermal growth factor receptor inhibitor, and external validation.

In accordance with an embodiment, the external validation may correspond to human intervention, such as scientists, researchers, subject matter experts, and case studies of some of the promising drug combinations that were prepared with literature evidence and mechanism of actions of both the drugs. In accordance with an exemplary embodiment, the determined one or more sets of drug combinations, which are novel, are shown in Table 3 below:

TABLE 3

| Drug Class Combination | Drug 1 | Drug 2 |
|---|---|---|
| Combination I | EGFR inhibitor | PTGS2 inhibitor |
| | Dacomitinib | (a) Sulindac |
| | | (b) Meloxicam |
| | | (c) Etodolac |
| | | (d) Naproxen |
| | | (e) Monobenzone |
| | | (f) Etoricoxib |
| | | (g) Rofecoxib |
| | | ₍h) Celecoxib |
| Combination II | EGFR inhibitor | ADRB2 inhibitor |
| | Dacomitinib | (a) Metoprolol |
| | | (b) Atenolol |
| | | (c) Doxofylline |
| | | (d) Propafenone |
| | | (e) Propranolol HCL |
| | | (f) Nadolol |
| | | (g) Nebivolol HCL |
| | | (h) Salmeterol Xinafoate |
| | | (i) Octreotide |
| | | (j) Timolol Maleate |
| | | (k) Carvedilol |

In accordance with an embodiment, a first pharmaceutical composition, such as Combination I, comprises an effective amount of Dacomitinib as EGFR inhibitor and PTGS2 inhibitor, and one or more pharmaceutically acceptable excipients. The PTGS2 inhibitor is selected from the group consisting of Sulindac, Meloxicam, Etodolac, Naproxen, Monobenzone, Etoricoxib, Rofecoxib, Celecoxib, or a pharmaceutically acceptable salt or prodrug thereof. The PTGS2 inhibitor may inhibit upregulated PTGS2 expression, which in turn increases the therapeutic effect of Dacomitinib in treatment of pancreatic cancer. Pharmacologic inhibition of PTGS2 sensitize the tumors to immunotherapy, suppress the growth of implanted tumors and increase the survival in pancreatic cancer tumor. Thus, Dacomitinib as EGFR inhibitor and the PTGS2 inhibitor, when combined in accordance with the first pharmaceutical composition, such as Combination I, produce synergistic effect in treating pancreatic cancer. In accordance with an embodiment, the pharmaceutical composition may be in the form of a first combination product.

In accordance with another embodiment, a second pharmaceutical composition, such as Combination II, comprises an effective amount of Dacomitinib as EGFR inhibitor and ADRB2 inhibitor, and one or more pharmaceutically acceptable excipients. The ADRB2 inhibitor is selected from the group consisting of Metoprolol, Atenolol, Doxofylline, Propafenone, Propranolol HCL, Nadolol, Nebivolol HCL, Salmeterol Xinafoate, Octreotide, Timolol Maleate, Carvedilol, or a pharmaceutically acceptable salt or prodrug thereof. The ADRB2 inhibitor may inhibit ADRB2 signaling that promotes cancer progression, which in turn increases the therapeutic effect of Dacomitinib in treatment of pancreatic cancer when administered in combination. Chronic stress hormones promote EGFR TKI resistance via β2-AR signaling and suggest the combinations of β-blockers with EGFR TKIs merit. Thus, Dacomitinib as an EGFR inhibitor and the ADRB2 inhibitor, when combined in accordance with the second pharmaceutical composition, such as Combination II, produce synergistic effects in treating pancreatic cancer. In accordance with an embodiment, the pharmaceutical composition may be in the form of a second combination product.

In accordance with an aspect of the present disclosure, a method of treating pancreatic cancer. In an embodiment, the method comprises the step of administering therapeutically effective amount of the first or the second pharmaceutical composition to an individual in need thereof, wherein the administration cures pancreatic cancer, thereby treating the individual. The pharmaceutical compositions disclosed herein may be administered to an individual in combination with other therapeutic compounds to increase the overall therapeutic effect of the treatment. The use of multiple compounds to treat an indication may increase the beneficial effects while reducing the presence of side effects.

Various routes of administration may be useful for administering therapeutically effective amounts of the first or the second pharmaceutical composition to an individual in need thereof, as disclosed herein, according to a method of treating pancreatic cancer disclosed herein. A pharmaceutical composition may be administered to an individual by any of a variety of means depending, for example, on the specific therapeutic compound or composition used, or other compound to be included in the composition, and the history, risk factors and symptoms of the individual. As such, topical, enteral, or parenteral routes of administration may be suitable for treating pancreatic cancer disclosed herein and such routes include both local and systemic delivery of a therapeutic compound or composition disclosed herein. Compositions comprising either a single therapeutic compound disclosed herein, or two or more therapeutic compounds disclosed herein are intended for inhaled, topical, intranasal, sublingual, intravenous, rectal and/or vaginal use may be prepared according to any method known to the art for the manufacture of pharmaceutical compositions.

In accordance with an embodiment, an individual is administered the first pharmaceutical composition comprising an effective amount of Dacomitinib as EGFR inhibitor and a PTGS2 inhibitor, and one or more pharmaceutically acceptable excipients. In accordance with another embodiment, the individual is administered a second pharmaceutical composition comprising an effective amount of Dacomitinib as EGFR inhibitor and an ADRB2 inhibitor, and one or more pharmaceutically acceptable excipients.

As used herein, the term “pharmaceutical composition” is synonymous with “pharmaceutically acceptable composition” or “pharmaceutically acceptable excipients” and refers to a therapeutically effective concentration of an active ingredient, such as, for example, any of the therapeutic compounds disclosed herein. As used herein, the term “pharmaceutically acceptable” refers to any molecular entity or composition that does not produce an adverse, allergic, or other untoward or unwanted reaction when administered to an individual. A pharmaceutical composition disclosed herein is useful for medical and veterinary applications. A pharmaceutical composition may be administered to an individual alone, or in combination with other supplementary active ingredients, agents, drugs, or hormones.

The pharmaceutical composition disclosed herein may comprise a therapeutic compound in a therapeutically effective amount. As used herein, the term “effective amount” is synonymous with “therapeutically effective amount”, “effective dose”, or “therapeutically effective dose” and when used in reference to treating pancreatic cancer refers to the minimum dose of a therapeutic compound disclosed herein necessary to achieve the desired therapeutic effect and includes a dose sufficient to treat pancreatic cancer. The effectiveness of a therapeutic compound disclosed herein in treating pancreatic cancer may be determined by observing an improvement in an individual based upon one or more clinical symptoms, and/or physiological indicators associated with the pancreatic cancer. Any improvement observed based on one or more clinical symptoms, and/or physiological indicators may be indicated by a reduced need for concurrent therapy.

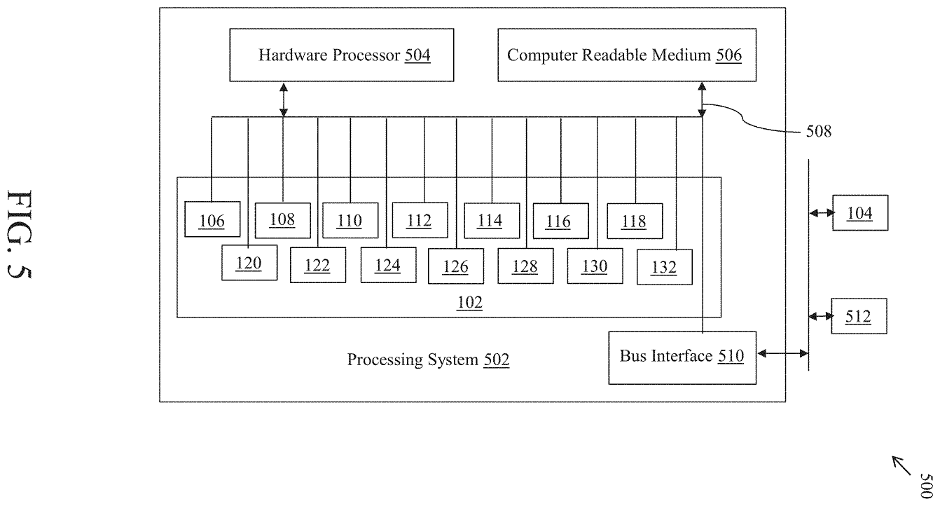

FIG. 5 is a conceptual diagram illustrating an example of a hardware implementation for a system employing a processing system for determining drug combinations and use in pancreatic cancer treatment, in accordance with an exemplary embodiment of the disclosure. Referring to FIG. 5, the hardware implementation shown by a representation 500 for the computing device 102 that employs a processing system 502 for determining drug combinations and use in pancreatic cancer treatment, as described herein.

In some examples, the processing system 502 may comprise one or more instances of a hardware processor 504, a non-transitory computer-readable medium 506, a bus 508, a bus interface 510, and a transceiver 512. FIG. 5 further illustrates the AI/ML engine 106, the processor 108, the dataset retrieval and normalization engine 110, the feature genes identification engine 112, the transcriptomics analysis engine 114, the target engine 116, the synergistic target engine 118, the drug combination engine 122, the scoring engine 120, the memory 124, the storage device 126, the input/output (I/O) device 128, the user interface 130, and the wireless transceiver 132, as described in detail in FIG. 1.

The hardware processor 504, such as the processor 108, may be configured to manage the bus 508 and general processing, including the execution of a set of instructions stored on the computer-readable medium 506. The set of instructions, when executed by the hardware processor 504, causes the computing device 102 to execute the various functions described herein for any particular apparatus. The hardware processor 504 may be implemented, based on several processor technologies known in the art. Examples of the hardware processor 504 may be RISC processor, ASIC processor, CISC processor, and/or other processors or control circuits.

The non-transitory computer-readable medium 506 may be used for storing data that is manipulated by the hardware processor 504 when executing the set of instructions. The data is stored for short periods or in the presence of power. The computer-readable medium 506 may also be configured to store data for one or more of the AI/ML engine 106, the processor 108, the dataset retrieval and normalization engine 110, the feature genes identification engine 112, the transcriptomics analysis engine 114, the target engine 116, the synergistic target engine 118, the scoring engine 120, and the drug combination engine 122.

The bus 508 may be configured to link together various circuits. In this example, the computing device 102 employing the processing system 502 and the non-transitory computer-readable medium 506 may be implemented with bus architecture, represented generally by bus 508. The bus 508 may include any number of interconnecting buses and bridges depending on the specific implementation of the computing device 102 and the overall design constraints. The bus interface 510 may be configured to provide an interface between the bus 508 and other circuits, such as, the transceiver 512, and external devices, such as the plurality of data sources 104.

The transceiver 512 may be configured to provide a communication of the computing device 102 with various other apparatus, such as the plurality of data sources 104, via a network. The transceiver 512 may communicate via wireless communication with networks, such as the Internet, the Intranet and/or a wireless network, such as a cellular telephone network, a wireless local area network (WLAN) and/or a metropolitan area network (MAN). The wireless communication may use any of a plurality of communication standards, protocols and technologies, such as 5th generation mobile network, Global System for Mobile Communications (GSM), Enhanced Data GSM Environment (EDGE), Long Term Evolution (LTE), wideband code division multiple access (W-CDMA), code division multiple access (CDMA), time division multiple access (TDMA), Bluetooth, Wireless Fidelity (Wi-Fi) (such as IEEE 802.11a, IEEE 802.11b, IEEE 802.11g and/or IEEE 802.11n), voice over Internet Protocol (VoIP), and/or Wi-MAX.

It should be recognized that, in some embodiments of the disclosure, one or more components of FIG. 5 may include software whose corresponding code may be executed by at least one processor, for across multiple processing environments. For example, the AI/ML engine 106, the processor 108, the dataset retrieval and normalization engine 110, the feature genes identification engine 112, the transcriptomics analysis engine 114, the target engine 116, the synergistic target engine 118, the scoring engine 120, and the drug combination engine 122, may include software that may be executed across a single or multiple processing environments.

In an aspect of the disclosure, the hardware processor 504, the non-transitory computer-readable medium 506, or a combination of both may be configured or otherwise specially programmed to execute the operations or functionality of the AI/ML engine 106, the processor 108, the dataset retrieval and normalization engine 110, the feature genes identification engine 112, the transcriptomics analysis engine 114, the target engine 116, the synergistic target engine 118, the scoring engine 120, and the drug combination engine 122, or various other components described herein, as described with respect to FIGS. 1 to 4.

Various embodiments of the disclosure comprise the computing device 102 that may be configured to determine drug combinations and use in pancreatic cancer treatment. The computing device 102 may comprise, for example, the AI/ML engine 106, the processor 108, the dataset retrieval and normalization engine 110, the feature genes identification engine 112, the transcriptomics analysis engine 114, the target engine 116, the synergistic target engine 118, the drug combination engine 122, the scoring engine 120, the memory 124, the storage device 126, the input/output (I/O) device 128, the user interface 130, and the wireless transceiver 132. One or more processors, such as the dataset retrieval and normalization engine 110, in the computing device 102 may be configured to retrieve pancreatic cancer datasets from a plurality of data sources based on selected types of expression profiling. One or more processors, such as the feature genes identification engine 112, in the computing device 102 may be configured to determine a set of feature genes based on differential gene expression analysis of disease samples and control samples in normalized pancreatic cancer datasets. One or more processors, such as the target engine 116, in the computing device 102 may be configured to select pancreatic cancer targets for combination analysis based on druggability and the determined set of feature genes. One or more processors, such as the synergistic target engine 118, in the computing device 102 may be configured to determine a plurality of synergistic target pairs based on node embedded clustering of the selected pancreatic cancer targets. One of each pair of target pairs is an epidermal growth factor receptor inhibitor. One or more processors, such as the drug combination engine 122, in the computing device 102 may be configured to select candidate pairs of drug combinations from a plurality of pairs of drug combinations based on a cumulative ranking score of each pair of drug combination and the plurality of synergistic target pairs. One or more processors, such as the drug combination engine 122, in the computing device 102 may be configured to determine one or more sets of drug combinations based on prioritization of the candidate pairs of drug combinations, filtration of drug combinations of the epidermal growth factor receptor inhibitor, and external validation.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

As utilized herein, the term “exemplary” means serving as a non-limiting example, instance, or illustration. As utilized herein, the terms “e.g.,” and “for example” set off lists of one or more non-limiting examples, instances, or illustrations. As utilized herein, circuitry is “operable” to perform a function whenever the circuitry comprises the necessary hardware and/or code (if any is necessary) to perform the function, regardless of whether performance of the function is disabled, or not enabled, by some user-configurable setting.

The terminology used herein is for the purpose of describing particular embodiments only and is not intended to be limiting of embodiments of the disclosure. As used herein, the singular forms “a”, “an” and “the” are intended to include the plural forms as well, unless the context clearly indicates otherwise. It will be further understood that the terms “comprises”, “comprising”, “includes” and/or “including”, when used herein, specify the presence of stated features, integers, steps, operations, elements, and/or components, but do not preclude the presence or addition of one or more other features, integers, steps, operations, elements, components, and/or groups thereof.

Further, many embodiments are described in terms of sequences of actions to be performed by, for example, elements of a computing device. It will be recognized that various actions described herein can be performed by specific circuits (e.g., application specific integrated circuits (ASICs)), by program instructions being executed by one or more processors, or by a combination of both. Additionally, these sequences of actions described herein can be considered to be embodied entirely within any non-transitory form of computer readable storage medium having stored therein a corresponding set of computer instructions that upon execution would cause an associated processor to perform the functionality described herein. Thus, the various aspects of the disclosure may be embodied in a number of different forms, all of which have been contemplated to be within the scope of the claimed subject matter. In addition, for each of the embodiments described herein, the corresponding form of any such embodiments may be described herein as, for example, “logic configured to” perform the described action.

Another embodiment of the disclosure may provide a non-transitory machine and/or computer-readable storage and/or media, having stored thereon, a machine code and/or a computer program having at least one code section executable by a machine and/or a computer, thereby causing the machine and/or computer to perform the steps as described herein for determining combination drug and use in pancreatic cancer treatment.

The present disclosure may also be embedded in a computer program product, which comprises all the features enabling the implementation of the methods described herein, and which when loaded in a computer system is able to carry out these methods. Computer program in the present context means any expression, in any language, code or notation, either statically or dynamically defined, of a set of instructions intended to cause a system having an information processing capability to perform a particular function either directly or after either or both of the following: a) conversion to another language, code or notation; b) reproduction in a different material form.

Further, those of skill in the art will appreciate that the various illustrative logical blocks, modules, circuits, algorithms, and/or steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present disclosure.

The methods, sequences and/or algorithms described in connection with the embodiments disclosed herein may be embodied directly in firmware, hardware, in a software module executed by a processor, or in a combination thereof. A software module may reside in RAM memory, flash memory, ROM memory, EPROM memory, EEPROM memory, registers, hard disk, physical and/or virtual disk, a removable disk, a CD-ROM, virtualized system or device such as a virtual server or container, or any other form of storage medium known in the art. An exemplary storage medium is communicatively coupled to the processor (including logic/code executing in the processor) such that the processor can read information from, and write information to, the storage medium. In the alternative, the storage medium may be integral to the processor.

While the present disclosure has been described with reference to certain embodiments, it will be noted understood by, for example, those skilled in the art that various changes and modifications could be made and equivalents may be substituted without departing from the scope of the present disclosure as defined, for example, in the appended claims. In addition, many modifications may be made to adapt a particular situation or material to the teachings of the present disclosure without departing from its scope. The functions, steps and/or actions of the method claims in accordance with the embodiments of the disclosure described herein need not be performed in any particular order. Furthermore, although elements of the disclosure may be described or claimed in the singular, the plural is contemplated unless limitation to the singular is explicitly stated. Therefore, it is intended that the present disclosure is not limited to the particular embodiment disclosed, but that the present disclosure will include all embodiments falling within the scope of the appended claims.

What is claimed is:

1. A pharmaceutical composition comprising an effective amount of Dacomitinib as epidermal growth factor receptor (EGFR) inhibitor and a prostaglandin-Endoperoxide Synthase 2 (PTGS2) inhibitor, and one or more pharmaceutically acceptable excipients.

2. The pharmaceutical composition according to claim 1, wherein the PTGS2 inhibitor is selected from the group consisting of Sulindac, Meloxicam, Etodolac, Naproxen, Monobenzone, Etoricoxib, Rofecoxib, Celecoxib, or a pharmaceutically acceptable salt or prodrug thereof.

3. The pharmaceutical composition according to claim 1 is in the form of a combination product.

4. The pharmaceutical composition according to claim 1, wherein the PTGS2 inhibitor inhibits upregulated PTGS2 expression which in turn increases the therapeutic effect of Dacomitinib in treatment of pancreatic cancer.

5. A pharmaceutical composition comprising an effective amount of Dacomitinib as epidermal growth factor receptor (EGFR) inhibitor and an Adrenoceptor Beta 2 (ADRB2) inhibitor, and one or more pharmaceutically acceptable excipients.

6. The pharmaceutical composition according to claim 5, wherein the ADRB2 inhibitor is selected from the group consisting of Metoprolol, Atenolol, Doxofylline, Propafenone, Propranolol HCL, Nadolol, Nebivolol HCL, Salmeterol Xinafoate, Octreotide, Timolol Maleate, Carvedilol, or a pharmaceutically acceptable salt or prodrug thereof.

7. The pharmaceutical composition according to claim 5 is in the form of a combination product.

8. The pharmaceutical composition according to claim 5, wherein the ADRB2 inhibitor inhibit ADRB2 signalling that promotes cancer progression, which in turn increases the therapeutic effect of Dacomitinib in treatment of pancreatic cancer.

9. A method of treating pancreatic cancer, the method comprising the step of administering a therapeutically effective amount of the pharmaceutical composition to an individual in need thereof, the pharmaceutical composition comprising an effective amount of Dacomitinib as epidermal growth factor receptor (EGFR) inhibitor with a prostaglandin-Endoperoxide Synthase 2 (PTGS2) inhibitor or an Adrenoceptor Beta 2 (ADRB2) inhibitor, and one or more pharmaceutically acceptable excipients.

10. The method of treating pancreatic cancer according to claim 9, wherein the PTGS2 inhibitor is selected from the group consisting of Sulindac, Meloxicam, Etodolac, Naproxen, Monobenzone, Etoricoxib, Rofecoxib, Celecoxib, or a pharmaceutically acceptable salt or prodrug thereof.

11. The method of treating pancreatic cancer according to claim 9, wherein the ADRB2 inhibitor is selected from the group consisting of Metoprolol, Atenolol, Doxofylline, Propafenone, Propranolol HCL, Nadolol, Nebivolol HCL, Salmeterol Xinafoate, Octreotide, Timolol Maleate, Carvedilol, or a pharmaceutically acceptable salt or prodrug thereof.

12. The method of treating pancreatic cancer according to claim 9, wherein the PTGS2 inhibitor inhibits upregulated PTGS2 expression, which in turn increases the therapeutic effect of Dacomitinib in treatment of pancreatic cancer.

13. The method of treating pancreatic cancer according to claim 9, wherein the ADRB2 inhibitor inhibit ADRB2 signalling that promotes cancer progression, which in turn increases the therapeutic effect of Dacomitinib in treatment of pancreatic cancer.
